(19) United States
(12) Patent Application Publication
(10) Pub. No.: US 2013/0140471 A1
(43) Pub. Date: Jun. 6, 2013

(54) Enhanced Output Mercury-Free UVC Lamp System

(71) Applicant: Robin Elkins, (US)

(72) Inventor: Robin Elkins, (US)

(73) Assignee: Quadra Intelligence, Chicago, IL (US)

(21) Appl. No.: 13/312,614

(22) Filed: Dec. 6, 2011

(51) Int. Cl. A61L 2/10; H01J 61/16; H01J 61/12

(52) U.S. Cl. 250/455.11; 313/572

(57) ABSTRACT

In overview, the described system provides a mercury-free gas discharge UVC lamp system and in particular a liquid purification system including a treatment container, a treatment volume, and one or more UVC lamps as described hereinafter configured to irradiate the treatment volume. In an embodiment of the invention, the UVC lamp system includes a tubular glass or quartz envelope, evacuated to approximately 200 Torr and slightly backfilled with a noble gas such as Xenon, Argon, or Nitrogen. For end emission, the cylindrical opening in each electrode assembly may be a through-hole capped externally by a quartz window. The radiation flux and dwell time may be adjusted to the type and degree of contamination in the water.

### TECHNICAL FIELD

This patent disclosure relates generally to production of electromagnetic radiation via discharge and, more particularly to a system and method of UVC production using a mercury free discharge lamp, as well as applications for the new system and method.

### BACKGROUND

Electrically powered lamps typically operate by flowing an electrical current through a medium. In the case of incandescent lights, the medium is a filament typically made of tungsten. The filament becomes hot, causing the thermal emission of photons. In the case of arc lamps, the medium may be a gas or gas mixture. Typically these lamps use mercury as part of the medium through which current flows, since the electronic state transmissions in mercury have proven to be of useful wavelengths. In particular, mercury vapor when excited can emit radiation in the UV range. When visible light is desired, a coating is used to interact with the UV radiation to produce visible radiation instead. This is the principle of operation, for example, in fluorescent lamps.

With respect to UV systems, the use of Mercury, while effective, is not without drawbacks. Mercury is known to be a human and environmental toxin, and both the production and disposal processes for mercury-containing lamps pose some rink. In addition, the accidental breakage of such lamps can impose a local contamination risk. For these reasons, the inventor feels that it is desirable to limit or eliminate the mercury used in UV systems.

This is especially true of UV systems used in disinfection or sterilization processes; these environments present a situation in which any contamination that may be present can have a significant negative impact on human health. Focusing further on the use of UV radiation in such situations, the UV portion of the electromagnetic spectrum includes radiation with a wavelength shorter than that of visible light, but longer than X-rays, in the range of 10 to 400 nm, with photon energies of about 3 eV to 124 eV. UV radiation can be classified into numerous different wavelength bands or energy bands. Three of these bands in the near and middle portion of the spectrum include UVA, UVB and UVC. UVA radiation has a fairly long wavelength at between 315 nm and 400 nm, and is sometimes referred to as “blacklight.” UVB is of a higher energy, and shorter wavelength at between 280 nm and 315 nm. Finally, UVC radiation is the most energetic of these classifications, with wavelengths between 100 nm and 280 nm. Of these categories of UV radiation, only UVC has substantial germicidal properties, since the DNA absorption peak is between about 240 nm and 280 nm and as such only UVC is fully effective in sterilization and purification.

Although some technologists have attempted to produce mercury free UVC systems for disinfection, such systems are complex and thus expensive and defect-prone, and as such, most lamp technologies continue to employ Mercury (or other metal vapor component). For example, US Pat. Appln. 2008/0284335 describes a Mercury-free lamp system for use in water disinfection. However, the lamp of the '335 application utilizes a complex system of coaxially nested electrodes with perforations to create an arc and a resultant plasma and radiation. Thus, there remains a need for a simple and effective UVC lamp for use in environments where heavy metal contamination is a concern.

It will be appreciated that this background description has been created by the inventors to aid the reader, and is not to be taken as a verbatim description of prior art, nor as an indication that any of the indicated problems were themselves appreciated in the art unless otherwise expressly indicated.

### SUMMARY

In overview, the described system provides in one embodiment a mercury-free gas discharge UVC lamp system and in particular a liquid purification system including a treatment container, a treatment volume, and one or more UVC lamps as described hereinafter configured to irradiate the treatment volume. The liquid may be flowed or static. In an embodiment of the invention, the UVC lamp system includes a tubular glass or quartz envelope, evacuated to approximately 200 Torr and slightly backfilled with a noble gas such as Xenon, Argon, or Nitrogen. Although in an alternative embodiment of the invention natural air could be used, this is not preferred.

The degree of backfilling is limited such that the final pressure within the envelope remains at or near about 200 Torr. The system media comprises the backfill gas alone, and does not contain Mercury or other materials or substrates. Although the physical systems used to allow evacuation and backfilling of the envelope are not critical, various means for such are provided in various embodiments of the invention. Thus, for example, the envelope may be evacuated and filled through a single opening, which is then sealed via deformation, crimping, etc. Alternatively, the envelope may be evacuated and filled through different openings. Moreover, although permanent sealing is preferred to maintain the integrity of the system, it is also possible in an embodiment of the invention to have a reversible seal such as a valve.

While the envelope is typically at least locally tubular in the described embodiments, the envelope need not be linear on a longer scale. In an embodiment it is contemplated that the envelope is wound in a spiral pattern, similar to the shape exhibited by a compact fluorescent light.

Although not a critical design feature, the ends of the envelope may be left open and bonded into corresponding cylindrical openings in respective electrode assemblies. For end emission, the cylindrical opening in each electrode assembly, or in at least one electrode assembly, is a through-hole capped externally by a quartz window. The quartz window is set at Brewster's angle in an embodiment of the invention to minimize internal reflection.

Once constructed and installed, an ionization voltage of approximately 15 kV is applied to the gas via the electrodes to cause the gas to ionize. Once ionized, a steady state current of about 30 mA is allowed to flow through the resultant plasma, at which time the voltage across the lamp drops to about 500 V. In an embodiment, the applied voltage is derived via a neon discharge lamp transformer, and may be further rectified, though it is not needed in most embodiments to further smooth the voltage before application to the lamp system.

It will be appreciated that the lamp may be operated continuously or in a pulsed mode. Taking into account the steady state voltage of the system and the plasma current, the steady state power consumption of the device is very low. The radiation output for the described system is primarily in the UVC portion of the spectrum, and in particular falls at about 248 nm. This wavelength is known to be biologically active, i.e., to affect the DNA and other aspects of microbial contaminants and prevent human infection by those microbial contaminants.

Numerous applications for this system are presented including but not limited to UVC water purification system. The contemplated water purification system has numerous embodiments depending upon the larger system, volume/flow rate requirements, and user preference. In one embodiment, numerous lamps as described above are placed so as to penetrate the treatment volume, such that lateral emission from each envelope interacts with the water being treated in a 360-degree treatment arc. The radiation flux and dwell time may be adjusted to the type and degree of contamination in the water.

It will be appreciated that different embodiments of the invention are not mutually exclusive, and elements from any embodiment may be combined with one or more elements from any other embodiment, and that the division of the discussion into embodiments is made in order to break the disclosure down for reader convenience and not to signify different mutually exclusive inventions. Further and alternative aspects and features of the disclosed principles will be appreciated from the following detailed description and the accompanying drawings, of which:

### BRIEF DESCRIPTION OF THE DRAWINGS

FIG. 1 is a schematic cross-sectional view of a UVC lamp configuration according to an embodiment of the invention;

FIG. 2 is a schematic cut away view of an alternative UVC lamp configuration according to an alternative embodiment of the invention;

FIG. 3 is a schematic cut away view of another alternative UVC lamp configuration according to a further alternative embodiment of the invention;

FIG. 4 is a schematic top view of an alternative UVC lamp configuration according to yet another alternative embodiment of the invention;

FIG. 5 is a system diagram showing a UVC lamp and associated operational and manufacturing aspects according to an embodiment of the invention; and

FIG. 6 is a cross-sectional top view of an exemplary water treatment configuration in an embodiment of the invention employing multiple UVC lamps according to the described principles.

### DETAILED DESCRIPTION

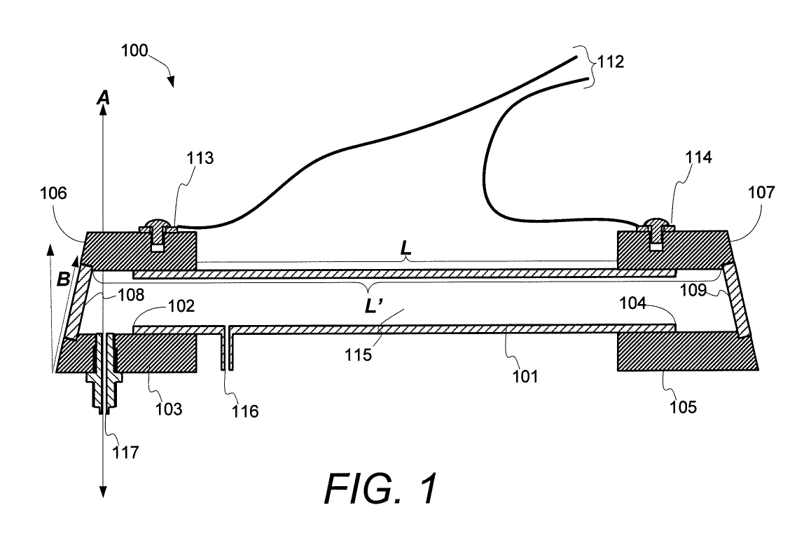

Turning now to the details of certain exemplary embodiments, FIG. 1 is a schematic cross-sectional view of a UVC discharge lamp according to an embodiment of the invention. In the illustrated embodiment of the invention, the UVC discharge lamp 100 comprises a UV-transparent envelope 101 in a generally tubular shape, having a length L. The material of the UV-transparent envelope 101 may be any suitable material including but not limited to “germicidal soft glass,” fused quartz glass, synthetic fused silica, etc.

The UV-transparent envelope 101 is of a hollow tubular shape with sufficient wall thickness to withstand atmospheric pressure when evacuated. Although the envelope may not be fully evacuated in its operational state, it may be substantially evacuated during manufacture to purge unwanted gases, as will be discussed in greater detail below.

The UV-transparent envelope 101 comprises a first end 102 and a second end 104 at opposite terminations of the UV-transparent envelope 101. The first end 102 of the UV-transparent envelope 101 is inserted into a first hollow end terminal 103, while the second end 104 of the UV-transparent envelope 101 is inserted into a second hollow end terminal 105. During manufacture, each end 102, 104 is sealingly bonded to the respective hollow end terminal 103, 105.

Each of the first hollow end terminal 103 and the second hollow end terminal 105 is formed with a terminating face, 106, 107 respectively, formed at Brewster's angle B for the expected radiation in an embodiment of the invention. It will be appreciated that the terminating faces 106, 107 of the first hollow end terminal 103 and the second hollow end terminal 105 may instead be formed at a right angle. Alternatively, only one of the first hollow end terminal 103 and the second hollow end terminal 105 may be terminated with a Brewster's angle in an alternative embodiment of the invention.

In an embodiment of the invention wherein end emission is desired, one or both of the terminating faces, 106, 107 comprises a countersink opening for holding a transparent window 108, 109. In the event that the respective transparent windows 108, 109 are situated some distance beyond the associated first end 102 and second end 104 of the UV-transparent envelope 101, the actual cavity length presented to radiation within the UV-transparent envelope 101 is L′, greater than L.

In an embodiment of the invention, each of the first hollow end terminal 103 and the second hollow end terminal 105 of the UV-transparent envelope 101 includes a terminal connection allowing a convenient access for electrical connectivity to the first hollow end terminal 103 and the second hollow end terminal 105. Cables 112 for supplying power to the first hollow end terminal 103 and the second hollow end terminal 105 may be connected to the terminal connections via connectors 113, 114.

As noted above, it is desirable to control the gas content of the cavity 115 defined by the UV-transparent envelope 101, the first and second hollow end terminals 103, 105, and the transparent windows 108, 109. To this end, in an embodiment of the invention, the cavity 115 is first evacuated and then slightly back-filled with an appropriate gas. This process will be discussed in greater detail below, but in order to facilitate the evacuation and back-filling described, a sealable inlet and outlet to the cavity 115 are required.

In an embodiment of the invention, the inlet and outlet are the same opening. In a further embodiment of the invention, the inlet/outlet comprises a branching connection 116 to the UV-transparent envelope 101. In this embodiment, the connection 116 may be used for both evacuation and backfilling, and may then be permanently closed via melting and pinching or other suitable technique as will be appreciated by those of skill in the art. In an alternative further embodiment of the invention, a threaded connecter 117 may be used for one or both of evacuation and back-filling, and may be sealably closed via a valve on the threaded connector itself or on a tube or pipe linked to the connector 117. It will be appreciated that multiple inlets and outlets may also be used, i.e., to provide an inlet that is at a different location or requires different connections than the outlet.

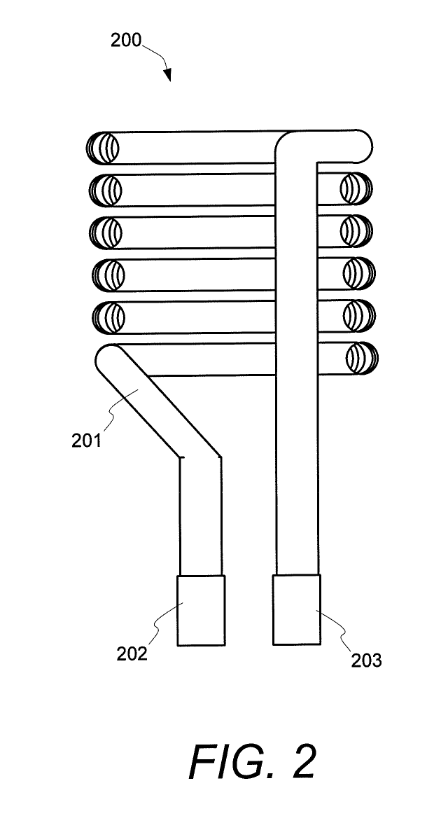

While the UV-transparent envelope 101 is preferably though not necessarily of a locally circular cross-section, it will be appreciated that the device need not be arranged in a linear configuration. For example, as shown in FIGS. 2-4, the device may be formed in a compact spiral, linear spiral, or looped configuration without departing from the scope of the described principles.

Referring specifically to the embodiment of the invention shown in FIG. 2, the illustrated UVC lamp 200 is configured in a compact spiral arrangement. In this configuration, the UV-transparent envelope 201 is coiled about a central cavity with a curvature suitable for the intended installation and material tolerances. An exemplary coil diameter in an embodiment of the invention is about four (4) inches, but the precise diameter is not critical, and much larger and much smaller dimensions may instead be used if desired.

The illustrated UVC lamp 200 in this embodiment of the invention 201 also comprises one terminal 202, 203 at each end of the UV-transparent envelope 201. The terminal in the illustrated embodiment of the invention is not hollow at the ends nor does it contain end windows, since the UVC lamp 200 in this embodiment is configured for radial emission and not for end emission. However, if desired, the device could be so configured without departing from the described principles.

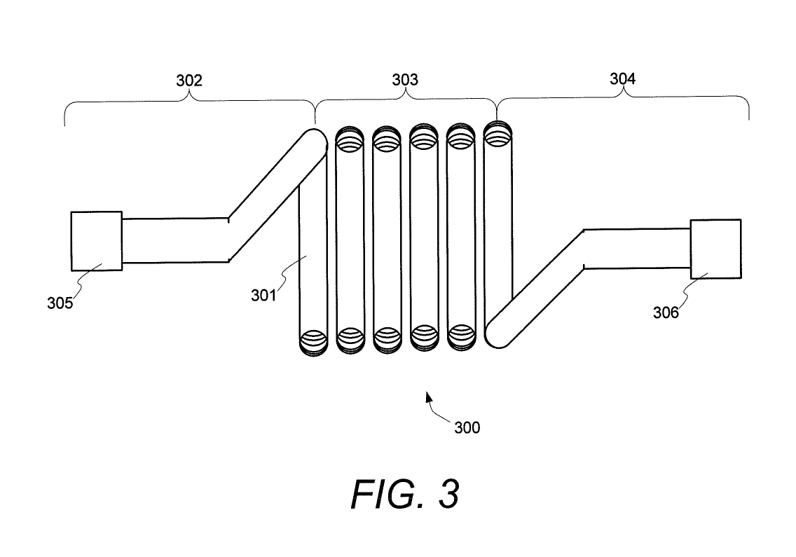

Referring to the embodiment of the invention illustrated in FIG. 3, the illustrated UVC lamp 300 is arranged in a linear spiral configuration. Thus, the UV-transparent envelope 301 transitions from a first horizontal run 302 to a horizontally extending spiral portion 303, and then to a second horizontal run 303. As with the compact spiral configuration discussed above with reference to FIG. 2, the illustrated UVC lamp 300 in this embodiment also comprises one terminal 305, 306 at each end of the UV-transparent envelope 301. Similarly, the terminals 305, 306 are not illustrated as being hollow, but may so configured without departing from the described principles if desired.

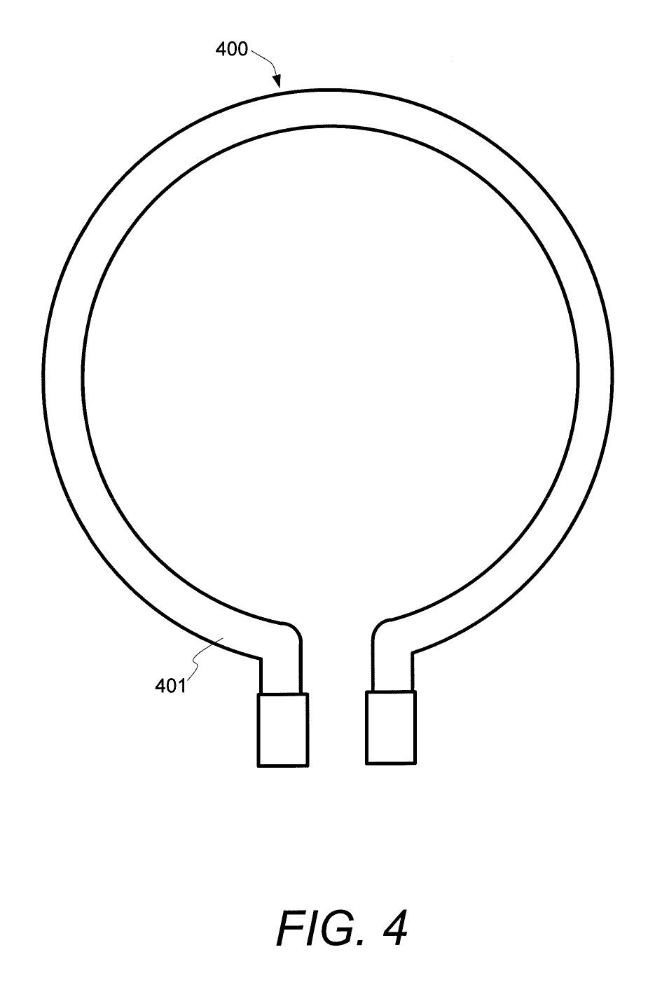

As shown in FIG. 4, a UVC lamp within the scope of the described principles may also be formed in a looped configuration. Thus, the illustrated UVC lamp 400 includes a UV-transparent envelope 401 that is formed substantially as an open circle. Where the circle opens, the UV-transparent envelope 401 is embedded in to respective terminals 402, 403 for connection to a suitable power source. With respect to this embodiment of the invention as well as other embodiments wherein the terminals are situated near one another, care is preferably taken to avoid contact or arcing between the terminals.

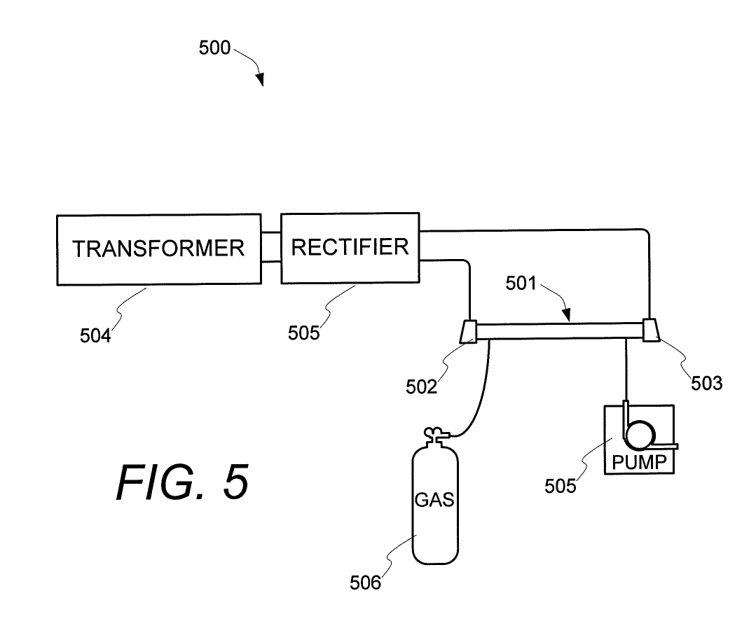

Having described various embodiments of the UVC lamp itself, FIG. 5 is directed to the overall system including the lamp, with components shown for both manufacture and use. It will be appreciated that the components for lamp manufacture and components for lamp use will not typically remain connected simultaneously, although they may be if desired.

In the illustrated configuration 500, the UVC lamp 501 is in a straight tubular configuration by way of example, but the illustrated arrangement can be applied as well to other configurations. The UVC lamp 501 as illustrated comprises two end terminals 502, 503 for receiving power for illumination of the lamp 501. A transformer 504 supplies an initial start voltage and a steady state voltage via a rectifier 505 to the lamp 501 via the end terminals 502, 503.

The rectifier 505 is preferably a full wave rectifier, although AC operation is also contemplated within the described principles. To this end, in an alternative embodiment of the invention, the output of the transformer 504 is provided directly to the terminals 502, 503 without rectification.

For evacuating the lamp 501, a vacuum pump 505 is connected to the lamp 501, and may be isolated from the lamp 501 after pumping. Means of isolation include valves (not shown) as well as melting/collapsing, crimping, etc. In order to backfill the envelope after evacuation, a gas source 506 is provided and linked to the lamp 501 via a suitable inlet, which as noted previously, need not be separate from the pumping inlet.

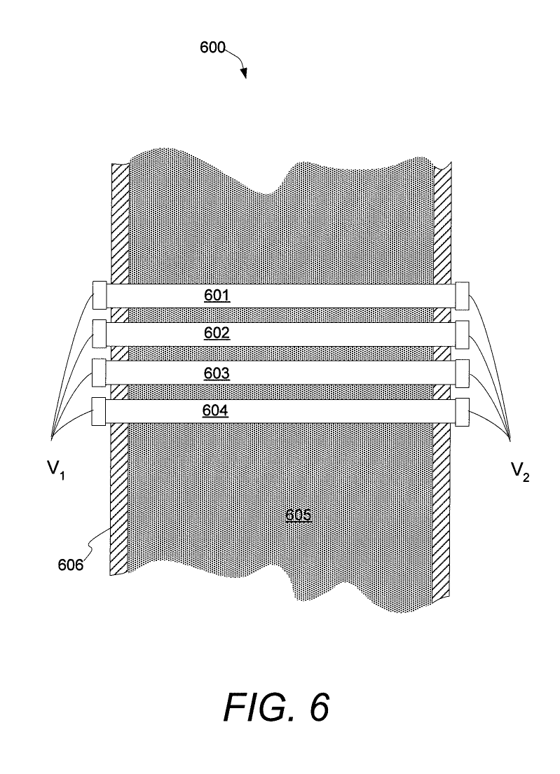

Having discussed the configuration, manufacture, and basic operating principles of the UVC lamp according to various embodiments of the invention, certain example application scenarios will now be discussed in greater detail. The schematic drawing of FIG. 6 shows a water treatment application 600 for the described UVC lamp, wherein waste water is flowed past one or more operating UVC lamps as described herein such that the emitted UVC radiation serves to purify the radiated water.

In particular, in the illustrated embodiment of the invention, a series of UVC lamps 601, 602, 603, 604 are arranged across a flow 605 of waste water or other liquid to be treated. The series of UVC lamps 601, 602, 603, 604 may be arranged in a plane, as illustrated, or may be arranged in a three-dimensional array depending upon the shape and volume of the treatment zone provided by the associated container 606. Each UVC lamp 601, 602, 603, 604 may be powered in parallel with the others as shown or alternatively may be powered independently.

It will be appreciated that the power terminals of the UVC lamps 601, 602, 603, 604 should be isolated from the associated container 606. It will be further appreciated that the envelopes or other suitable surfaces of the UVC lamps 601, 602, 603, 604 should be sealed against the proximate surfaces of the associated container 606 to restrict the fluid flow 605 to the volume within the container.

Returning to FIG. 1 to discuss the operating principles of the UVC lamp system 100, the envelope 101 has been evacuated and backfilled to approximately 200 Torr with a noble gas such as Xenon, Argon, or Nitrogen. As made, the system media comprises the backfill gas alone, and does not contain Mercury or other materials or substrates within the envelope 101. Once constructed and installed, an ionization voltage of about 15 kV is applied to the encapsulated gas via the electrodes, causing the gas to ionize.

Once the encapsulated gas ionized, a steady state current of about 30 mA is allowed to flow through the resultant plasma, at which time the voltage across the lamp drops to a steady state voltage of about 500 V. As previously noted, the lamp may be operated continuously or in a pulsed mode. The discontinuous emission is pulsed mode tends to have a higher peak power, which may be desired if the material being treated exhibits a power-dependent non-cumulative response. The radiation output for the system as described is primarily in the UVC portion of the spectrum, and in particular falls at about 248 nm, a wavelength that is biologically active, and hence useful in deactivating microbial contaminants.

Numerous applications for this system are possible beyond the described system for UVC water purification. For example, the disclosed system, when used with one or more hollow electrodes and associated windows provides an output of ASE or LASER radiation. ASE, or Amplified Spontaneous Emission, occurs when a laser gain medium is in a state of population inversion. While ASE is not the same as LASER emission, the feedback of ASE via the optical cavity may produce laser operation if the lasing threshold is reached. In that case the ASE, which was not necessarily coherent radiation, can produce coherent LASER propagation. Although the linear lamps described contain the necessary inverted medium and can allow a standing wave oscillation, there are many other arrangements which may also be used, including traveling wave oscillators such as ring LASERS. In an embodiment of the invention, a spark gap can be introduced in one leg of the circuit coming from a capacitor, to ensure the rapid discharge through the lamp (from the capacitor, which has stored high voltage and high energy), for (short) pulsed mode operation.

In more general terms, it will be appreciated that the foregoing description provides useful examples of the disclosed system and technique. However, it is contemplated that other implementations of the disclosure may differ in detail from the foregoing examples. All references to the disclosure or examples thereof are intended to reference the particular example being discussed at that point and are not intended to imply any limitation as to the scope of the disclosure more generally. All language of distinction and disparagement with respect to certain features is intended to indicate a lack of preference for the features of interest, but not to exclude such from the scope of the disclosure entirely unless otherwise specifically indicated.

We claim:

1. A mercury-metal free lamp for emission of UVC radiation comprising:
a gas tight at least partially UVC-transparent envelope for submersion in water, the envelope having a substantially circular cross section and a central axis;
an electrode sealing each end of the gas tight envelope; and
an arc medium within the gas tight envelope, the arc medium comprising a gas at a pressure of about 200 Torr or less, such that energization of the electrodes at sufficient voltage will initiate an arc substantially along the envelope axis resulting in UVC emission within the envelope, with at least a portion of the generated UVC radiation passing through the envelope and into the water.

2. The mercury-metal free lamp for emission of UVC radiation in accordance with claim 1, wherein the gas tight envelope is in a linear cylindrical configuration.

3. The mercury-metal free lamp for emission of UVC radiation in accordance with claim 1, wherein the gas tight envelope is in a spiral configuration.

4. The mercury-metal free lamp for emission of UVC radiation in accordance with claim 1, wherein the arc medium is Xenon.

5. The mercury-metal free lamp for emission of UVC radiation in accordance with claim 1, wherein the arc medium is Argon.

6. The mercury-metal free lamp for emission of UVC radiation in accordance with claim 1, wherein the arc medium is Nitrogen.

7. The mercury-metal free lamp for emission of UVC radiation in accordance with claim 1, wherein energization of the electrodes for creating an arc is about 15 kV.

8. The mercury-metal free lamp for emission of UVC radiation in accordance with claim 1, wherein the lamp requires a steady state current of about 30 mA or less and a steady state voltage of about 500 V or less to provide a steady state UVC output.

9. The mercury-metal free lamp for emission of UVC radiation in accordance with claim 1, wherein the lamp further comprises at least one UVC-transparent window at one end of the gas tight envelope, and wherein the envelope length is such that energization of the electrodes results in ASE output through the UVC-transparent window.

10. The mercury-metal free lamp for emission of UVC radiation in accordance with claim 1, wherein the lamp further comprises at least one UVC-transparent window at one end of the gas tight envelope, and wherein the envelope length is such that energization of the electrodes results in LASER output through the UVC-transparent window.

11. A water treatment facility comprising:
a treatment volume having therein an amount of a fluid; and
a UVC emission device for radiating the amount of fluid with UVC radiation o sterilize the fluid, the UVC emission device further comprising:
a UVC lamp having a gas tight at least partially UVC-transparent envelope submerged in the fluid in the treatment volume, the envelope having a substantially circular cross section and a central axis, an electrode sealing each end of the gas tight envelope, and a gaseous arc medium within the gas tight envelope, the arc medium comprising a gas at a pressure of about 200 Torr or less, such that energization of the electrodes at sufficient voltage will initiate an arc substantially along the envelope axis resulting in UVC emission within the envelope, with at least a portion of the generated UVC radiation passing through the envelope and into the fluid

12. The water treatment facility according to claim 11, wherein the fluid is primarily water.

13. The water treatment facility according to claim 11, wherein the UVC emission device comprises multiple such UVC lamps.

14. The water treatment facility in accordance with claim 11, wherein the gaseous arc medium is Xenon.

15. The water treatment facility in accordance with claim 11, wherein the gaseous arc medium is Argon.

16. The water treatment facility in accordance with claim 11, wherein the gaseous arc medium is Nitrogen.

17. The water treatment facility in accordance with claim 11, wherein energization of the electrodes for creating an arc is about 15 kV.

18. The water treatment facility in accordance with claim 11, wherein the lamp requires a steady state current of about 30 mA or less and a steady state voltage of about 500 V or less to provide a steady state UVC output.
